(19) United States
(12) Patent Application Publication
(10) Pub. No.: US 2012/0116535 A1
(43) Pub. Date: May 10, 2012

(54) TELESCOPIC PROSTHESIS

(76) Inventors: Yves-Alain RATRON, Grenoble (FR); Irene FERRARI, Le Touvet (FR)

(21) Appl. No.: 13/155,209

(22) Filed: Jun. 7, 2011

Related U.S. Application Data
(60) Provisional application No. 61/352,702, filed on Jun. 8, 2010.

(30) Foreign Application Priority Data
Jun. 7, 2010 (FR) 1054448
Dec. 17, 2010 (FR) 1004933

(51) Int. Cl. A61F 2/30

(52) U.S. Cl. 623/23.45

(57) ABSTRACT

A modular prosthesis according to embodiments of the present invention includes a diaphyseal element adapted for implantation into a bone, a metaphyseal element with a mount adapted to receive a prosthetic articulating surface, a fluid reception chamber formed between the diaphyseal element and the metaphyseal element, the fluid reception chamber having proximal and distal ends and a side wall, one end being formed by the diaphyseal element and the other end being formed by the metaphyseal element, and an injection canal in fluid communication with the fluid reception chamber, wherein the fluid reception chamber is configured to receive fluid injected via the injection canal, such that a length of the modular prosthesis is adjustable in a longitudinal dimension based on a volume of fluid received by the fluid reception chamber. Progressive insertion of agglutinating fluid into the fluid reception chamber lengthens the prosthesis.

### CROSS-REFERENCE TO RELATED APPLICATIONS

This application claims the benefit of U.S. Provisional Patent Application Ser. No. 61/352,702, filed on Jun. 8, 2010, and also claims foreign priority to French Patent Application No. FR1054448, filed on Jun. 7, 2010 and to French Patent Application No. FR1004933, filed on Dec. 17, 2010. Each of these three applications is incorporated herein by reference in its entirety for all purposes.

### TECHNICAL FIELD

Embodiments of the present invention relate generally to systems and methods for modular prostheses, and more specifically to prostheses with adjustability as between their metaphyseal and diaphyseal portions.

### BACKGROUND

Existing unitary prostheses are implanted in a rigid manner, usually with the use of bone cement, but they often require the use of trial implants and are difficult to adjust. Existing modular prostheses offer a greater flexibility for adjustment, but present several disadvantages; for example, existing modular prostheses are often fragile, risk breaking, and risk experiencing relative displacement between the modular elements.

### SUMMARY

Embodiments of the present invention include modular prostheses which offer both ease of adjustment and also rigidity of fixation.

A modular prosthesis according to embodiments of the present invention includes a diaphyseal element adapted for implantation into a bone, a metaphyseal element including a mount, the mount adapted to receive a prosthetic articulating surface, a fluid reception chamber formed between the diaphyseal element and the metaphyseal element, the fluid reception chamber including a proximal end, a distal end, and a side wall, wherein one of the proximal end and the distal end is formed by the diaphyseal element, wherein the other of the proximal end and distal end is formed by the metaphyseal element; and an injection canal in fluid communication with the fluid reception chamber, wherein the fluid reception chamber is configured to receive fluid injected via the injection canal, such that a length of the modular prosthesis is adjustable in a longitudinal dimension based on a volume of fluid received by the fluid reception chamber.

According to some embodiments of the present invention, the side wall and/or the proximal end are formed by the metaphyseal element. In some cases, the injection canal may be substantially cylindrical and formed about a canal axis, the fluid reception chamber may be substantially cylindrical and formed about a chamber axis, and the chamber axis and canal axis may be substantially coaxial. The injection canal may be formed in the metaphyseal element.

According to some embodiments of the present invention, the diaphyseal element includes a proximal end and a distal end, the proximal end of the diaphyseal element being substantially cylindrical and having an outer diameter, the side wall being substantially cylindrical and having an inner diameter, wherein the outer diameter is larger than the inner diameter. In some cases, the metaphyseal element is adjustable relative to the diaphyseal element along a transverse dimension perpendicular to the longitudinal dimension, prior to hardening of the fluid. The metaphyseal element may be rotatably adjustable relative to the diaphyseal element about an axis substantially parallel to the longitudinal dimension, prior to hardening of the fluid.

According to some embodiments of the present invention, the diaphyseal element includes a proximal portion and a distal portion, wherein the distal portion is implanted into the bone, wherein the metaphyseal element includes the side wall, wherein the side wall includes a seal cavity, the modular prosthesis further including a seal housed by the seal cavity, an inner surface of the seal conforming to an outer surface of the proximal portion of the diaphyseal element, wherein the proximal portion slides longitudinally with respect to the seal while the seal substantially prevents escape of the fluid from the fluid reception chamber beyond the seal.

In some instances of embodiments, the seal cavity has a longitudinal height, the seal substantially fills the longitudinal height, and the seal cavity has an inner transverse dimension larger than an outer transverse dimension of the seal, such that the seal maintains a fluid barrier when the metaphyseal element is adjusted relative to the diaphyseal element along a transverse dimension perpendicular to the longitudinal dimension, prior to hardening of the fluid. A distal portion of the diaphyseal element may be implanted into the bone, and the proximal portion interlocks with the metaphyseal element to substantially inhibit rotation of the metaphyseal element with respect to the diaphyseal element about the longitudinal dimension, according to embodiments of the present invention. In some cases, the diaphyseal element includes a shoulder located between its proximal and distal ends.

A method for implanting a modular prosthesis according to embodiments of the present invention includes inserting an agglutinating fluid progressively into a fluid reception chamber formed between a first prosthesis element and a second prosthesis element in order to adjust a position of the first prosthesis element with respect to the second prosthesis element along a longitudinal dimension, and permitting the agglutinating fluid to agglutinate to rigidly fix positioning of the first prosthesis element with respect to the second prosthesis element. According to such embodiments of methods, a first volume of the fluid reception chamber may be variable, and inserting the agglutinating fluid progressively into the fluid reception chamber includes increasing at least one dimension of the fluid reception chamber based on a second volume of agglutinating fluid inserted into the fluid reception chamber. Such methods according to embodiments of the present invention may further include adjusting the position of the first prosthesis element with respect to the second prosthesis element along a transverse dimension substantially perpendicular to the longitudinal dimension prior to agglutination of the agglutinating fluid, and/or adjusting the position of the first prosthesis element with respect to the second prosthesis element rotationally about an axis corresponding to the longitudinal dimension prior to agglutination of the agglutinating fluid.

Inserting agglutinating fluid may include inserting the agglutinating fluid progressively into the fluid reception chamber via an injection channel formed in one or more of the first prosthesis element and the second prosthesis element. For example, the injection channel may be formed in the first prosthesis element.

Such methods according to embodiments of the present invention may further include forming a bone cavity in a bone (e.g. the intramedullary canal of a fractured or damaged humerus or femur), placing the second prosthesis element into the bone cavity; and rigidly fixing the second prosthesis element to the bone cavity with surgical cement. In some cases, rigidly fixing the second prosthesis element to the bone cavity is done before adjusting the position of the first prosthesis element with respect to the second prosthesis element.

A modular prosthesis, comprising a first element and a second element, wherein at least one fluid reception chamber is formed at an interface between the first element and the second element.

The modular prosthesis of paragraph [0013], wherein the at least one fluid reception chamber comprises a volume that is variable based on a quantity of fluid admitted into the at least one fluid reception chamber.

The modular prosthesis of paragraph [0013], wherein the at least one fluid reception chamber is a plurality of fluid reception chambers formed in an interior of the modular prosthesis, the plurality of fluid reception chambers being adapted to be filled independently of each other.

The modular prosthesis of paragraph [0013], wherein a relative positioning between the first element and the second element is axially variable along a longitudinal axis of the modular prosthesis.

The modular prosthesis of paragraph [0013], wherein a relative positioning between the first element and the second element is transversally variable with respect to a longitudinal axis of the modular prosthesis.

The modular prosthesis of paragraph [0013], wherein a relative positioning between the first element and the second element is angularly variable about a longitudinal axis of the modular prosthesis.

The modular prosthesis of paragraph [0013], wherein at least one of the first element and the second element includes an injection canal for injection of fluid into the at least one fluid reception chamber.

The modular prosthesis of paragraph [0019], wherein the second element includes at least one coupling cavity adapted to at least partially receive the first element, and wherein the at least one fluid reception chamber is formed in the coupling cavity.

The modular prosthesis of paragraph [0020], wherein a proximal portion of the first element and the coupling cavity of the second element comprise complementary locking elements, wherein the complementary locking elements are adapted to cooperate and lock relative rotational movement between the first and second elements.

The modular prosthesis of paragraph [0021], wherein the complementary locking elements comprise one or more of grooves, cannulations, teeth, and surface bumps.

The modular prosthesis of paragraph [0020], wherein the coupling cavity, the injection canal, and the first element are coaxial.

The modular prosthesis of paragraph [0013], further comprising a shoulder located between a proximal portion and a distal portion of the first element.

The modular prosthesis of paragraph [0013], wherein one of the first and second elements comprises an annular reception cavity located between the at least one fluid reception chamber and the other of the first and second elements, the modular prosthesis further comprising an annular watertight seal positioned in the annular reception cavity in order to seal the at least one fluid reception chamber with respect to the other of the first and second elements.

The modular prosthesis of paragraph [0013], wherein the at least one fluid reception chamber is configured to receive agglutinant fluid into direct contact with the first and second elements.

The modular prosthesis of paragraph [0013], further comprising an agglutinant fluid filling the at least one fluid reception chamber.

The modular prosthesis of paragraph [0027], wherein the agglutinant fluid is selected from the group consisting of: poly(methyl methacrylate) (PMMA), biocompatible resin, adhesive material, polymeric material, surgical cement, and an injectable bone substitute.

The modular prosthesis of paragraph [0027], wherein the agglutinant fluid has a drying time from five to thirty minutes.

The modular prosthesis of paragraph [0013], wherein the fluid is a non-agglutinant fluid selected from the group consisting of: air, water, liquid solution, and aqueous gel.

The modular prosthesis of paragraph [0013], wherein the second second element is a first second element, the surgical kit comprising a second element, wherein each of the first and second second elements comprises different dimensions and is configured to interface with the first element to form a fluid reception chamber of differing dimensions.

While multiple embodiments are disclosed, still other embodiments of the present invention will become apparent to those skilled in the art from the following detailed description, which shows and describes illustrative embodiments of the invention. Accordingly, the drawings and detailed description are to be regarded as illustrative in nature and not restrictive.

### BRIEF DESCRIPTION OF THE DRAWINGS

FIG. 1 illustrates a front elevation cross sectional view of a modular prosthesis implanted in a fractured bone, according to embodiments of the present invention.

FIG. 2 illustrates a front elevation cross section view of another modular prosthesis, according to embodiments of the present invention.

FIG. 3 illustrates a front elevation view of a variation of a diaphyseal element of a modular prosthesis, according to embodiments of the present invention.

FIG. 4 illustrates a front elevation cross section view of another modular prosthesis, according to embodiments of the present invention.

FIG. 5 illustrates an enlarged view of a portion of the modular prosthesis of FIG. 4 taken from detail region V of FIG. 4, according to embodiments of the present invention.

FIG. 6 illustrates a front elevation cross section view of the modular prosthesis of FIGS. 4 and 5 in an alternative configuration, according to embodiments of the present invention.

While the invention is amenable to various modifications and alternative forms, specific embodiments have been shown by way of example in the drawings and are described in detail below. The intention, however, is not to limit the invention to the particular embodiments described. On the contrary, the invention is intended to cover all modifications, equivalents, and alternatives falling within the scope of the invention as defined by the appended claims.

### DETAILED DESCRIPTION

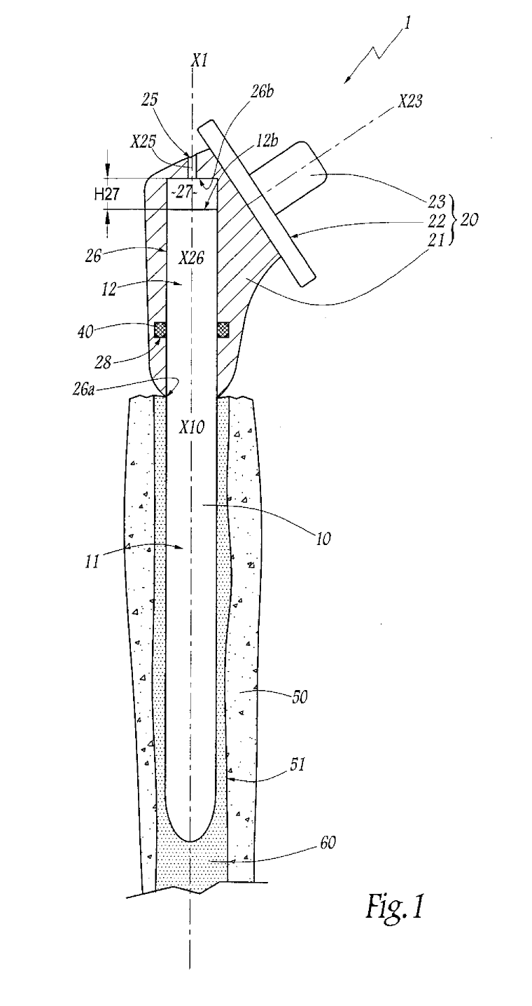

FIG. 1 illustrates a modular prosthesis 1 which extends substantially along an axis X1, including a diaphyseal element 10 and a metaphyseal element 20, according to embodiments of the present invention. The prosthesis 1 is adapted to be implanted in an intramedullary cavity 51, which is prepared by the surgeon in a bone 50. In particular, the bone is a humeral diaphysis 50, and the surgeon implants the prosthesis 1 with the goal of repairing a shoulder fracture, according to embodiments of the present invention.

The diaphyseal element is a cylindrical rod 10 including a distal portion 11 and a proximal portion 12, which extend along longitudinal axis X10. During implantation of the prosthesis 1 in the diaphysis 50, the rod 10 is first inserted into the cavity 51 of the diaphysis 50. In order to lock the rod 10 in its final configuration, cement or resin 60 is injected in liquid form in the cavity 51. In this way, after hardening of the cement or the resin, the distal portion 11 of the rod is rigidly fixed in the cavity 51 of the diaphysis 50, and the proximal portion 12 projects beyond the cavity 51. On its end opposite the cavity 51, the proximal portion 12 includes a proximal end 12b in the form of a disc, according to embodiments of the present invention. Although a rod is shown, the diaphyseal element may instead by a nail, for example an intramedullary nail.

The metaphyseal element 20 includes a head 21, a support surface 22, and a mount 23. The support surface 22 and the mount 23 serve as a support for a ball portion of a ball-and-socket joint, or another articulating surface (not shown). The mount 23 has a cylindrical form, and extends perpendicularly from the support surface 22 along an axis X23, according to embodiments of the present invention.

The metaphyseal element 20 is made by known molding, fabrication, drilling, and machining techniques, and the metaphyseal element 20 of FIG. 1 is unitary, or in other words is formed integrally as a single piece of material. In another embodiment (not shown), the support surface 22 and the mount 23 are rigidly coupled to the head 21 rather than being unitary with the head 21.

The head 21 is partially hollow and includes: an injection canal 25 extending along a longitudinal axis X25, a coupling cavity 26 extending along a longitudinal axis X26 and in which is formed a fluid reception chamber 27, and a cavity 28 for receiving an annular seal 40. The injection canal 25 and the cavity 26 are cylindrical, and may be coaxial, according to embodiments of the present invention. This facilitates their formation into the head 21, for example with drilling. The cavity 26 forms a mouth opening 26A next to the diaphyseal cavity 51, as well as an internal wall 26b which extends perpendicularly and coaxially to the axis X26 and at the level at which the canal 25 opens, according to embodiments of the present invention. In an alternative embodiment (not shown), the injection canal 25 and the cavity 26 are not coaxial.

In practice, the metaphyseal element 20 is positioned by the surgeon on the rod 10, which serves as intermediate support between the metaphyseal element 20 and the bone 50. More specifically, the proximal portion 12 of the rod 10 penetrates into the coupling cavity 26, with sliding of the complementary cylindrical walls. In this way, the chamber 27, which is formed by cylindrical walls of the cavity 26, the surface 26b and the end 12b of the proximal portion 12, has a variable volume. In particular, the reception chamber 27 has a variable dimension H27 along the longitudinal direction of the prosthesis 1, defined by its axis X1, with which the axis X10 of the rod 10 overlaps. This dimension H27 may be measured along axis X1 between surfaces 12b and 26b, according to embodiments of the present invention.

At this stage, the height of the prosthesis 1 can be adjusted by progressively injecting a fluid (not shown) through the injection canal 25 and into the reception chamber 27. The fluid is injected at the interface between the rod 10 and the metaphyseal element 20, into direct contact with the rod 10 and the metaphyseal element 20, and not into contact with any other element in the chamber 27 according to embodiments of the present invention. The rod 10 is rigidly fixed in the diaphyseal cavity, whereas the metaphyseal element 20 can be displaced relative to the proximal element 12, according to embodiments of the present invention. Under the effect of the internal pressure in the chamber 27, exerted by the fluid between the cavity 26 and the proximal portion 12, the volume of the chamber 27 increases. Specifically, the dimension H27 of the fluid reception chamber 27 is variable, along the axis X1, as a function of the quantity of fluid admitted into the chamber 27. Accordingly, as the fluid is injected, the height of the prosthesis 1 increases progressively until attaining a desired position, according to embodiments of the present invention. In one alternative embodiment (not shown), the metaphyseal element 20 is provided with a visualization mechanism to indicate the height of the prosthesis, for example graduation marks on an external surface.

The fluid used may be an agglutinant. As such, during the acceptance of the agglutinant fluid, in particular as it dries, the modular elements 10 and 20 are locked with respect to each other in the chosen position, in translation and in rotation, with a selected height. In other words, the particular configuration of the modular prosthesis 1 and the injection of an agglutinant fluid permits the telescopic adjustment of the height. Furthermore, the relative angular positioning between the elements 10 and 20, about the longitudinal axis X1, is variable and can be adjusted with precision before, during, or after the injection of the agglutinant fluid, according to embodiments of the present invention.

As used herein, an agglutinating fluid is a material susceptible to agglutination between two elements with which it finds itself in contact, so as to reunite them in a unitary combination, to join them in a way that prevents all relative movement, according to embodiments of the present invention. This material is fluid as it is introduced into the reception chamber 27, then dries progressively until it hardens, in order to become a solid hardened volume in the implanted prosthesis.

According to some embodiments of the present invention, the agglutinant fluid one or more of a poly(methyl methacrylate) (PMMA), a biocompatible resin, an adhesive material, a polymeric material, a surgical cement, and/or an injectable bone substitute. The agglutinant fluid may be, for example, initially contained in a deformable container including an injection conduit, or may be injected with a manual gun, according to embodiments of the present invention. Successive injections can be made until the desired height is attained, then the drying of the agglutinant fluid permits the locking of the modular elements with respect to each other, at the level of the coupling cavity 26 and the reception chamber 27, according to embodiments of the present invention. The drying time of the agglutinant fluid is between five and thirty minutes, according to embodiments of the present invention.

Moreover, a de-agglutinant or fluidizing fluid may be alternatively injected into the reception chamber 27, according to embodiments of the present invention. Such a fluid permits the dissolving or the refluidification of the agglutinant fluid, in order to modify the relative position of the prosthetic elements, for example during a post-operative intervention, according to embodiments of the present invention.

The proximal portion 12 of the rod 10 is adjusted in the cavity 26, however the presence of the annular seal 40 eventually permits completion of the watertightness of the chamber 27 and the cavity 26 on the side oriented toward the diaphysis 50, to prevent the agglutinant fluid from escaping from the chamber 27 and cavity 26, according to embodiments of the present invention.

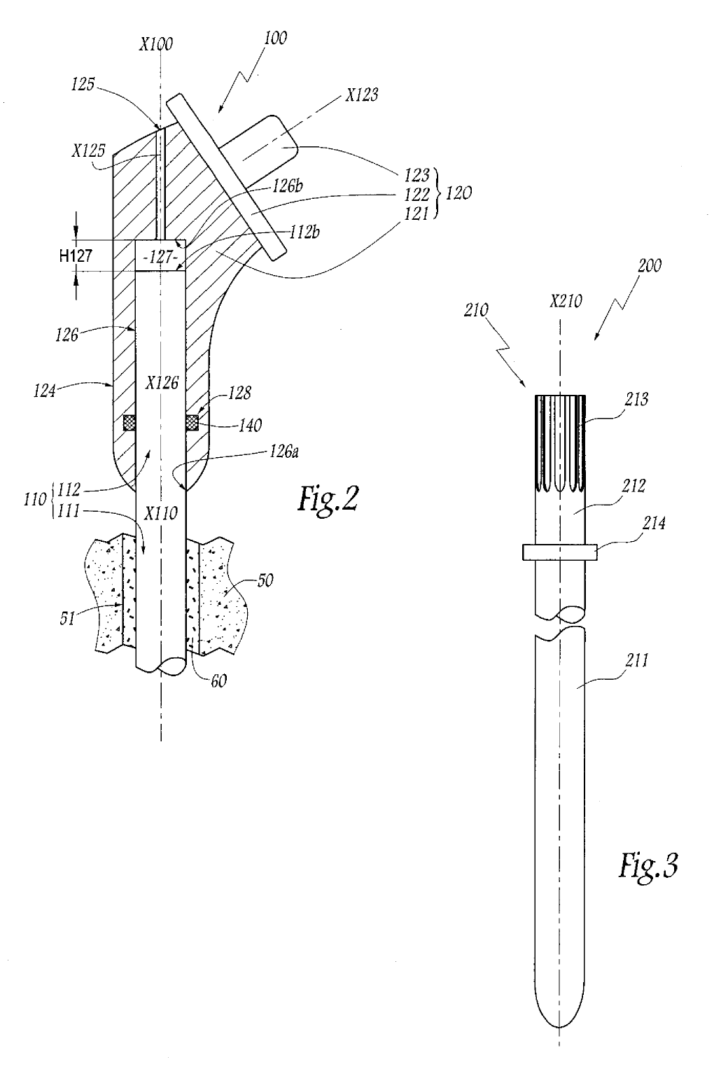

FIG. 2 illustrates an alternative modular prosthesis 100, according to embodiments of the present invention. Prosthesis 100 is adapted to be positioned in the same bone 50 as the prosthesis 1, according to embodiments of the present invention.

Certain elements of the prosthesis 100 are similar to the elements of prosthesis 1, described above, and are labeled with the same numbers increased by 100. This includes the rod 110 with axis X110 including a distal portion 111 and a proximal portion 112 with end 112b, the support surface 122, the mount 123 with axis X123, the injection canal 125 with axis X125, the coupling cavity 126 with axis X126, the mouth 126a and an interior surface 126b, the cavity 128 and the annular seal 140, according to embodiments of the present invention.

The dimensions of the metaphyseal element 120 are different from the dimensions of the metaphyseal element 20. In particular, the head 121 of the metaphyseal element 120 includes particular internal dimensions and arrangement, for example a distal portion 124 elongated in which extends the coupling cavity 126 and the elongated injection canal 125. This configuration permits, for a range of adjustment in height identical to the height adjustment range of prosthesis 1, the surgeon to access different prosthesis dimensions than those of prosthesis 1, according to embodiments of the present invention. In fact, if the range of adjustment in height of the prosthesis 1 was found insufficient to obtain optimal adjustment, the configuration of prosthesis 100 may be employed to obtain the optimal adjustment. As with prosthesis 1, the dimension H127 of the reception chamber 127 is variable as a function of the quantity of the fluid admitted into the chamber 127, according to embodiments of the present invention. In this way, because of the configuration of the chamber 127 and the elongated portion 124, the height to which the prosthesis 100 can be adjusted is larger than that of the prosthesis 1.

In practice, a range of metaphyseal elements 20, 120, and others, can be provided to cover different height ranges, so as to best respond to the needs of the surgeon. Each metaphyseal element may cover approximately a range of fifteen millimeters in height, according to embodiments of the present invention.

FIG. 3 illustrates another alternative modular prosthesis 200, according to embodiments of the present invention. Prosthesis 200 includes a variation of the diaphyseal rod 210. The cylindrical rod 210 includes a distal portion 211 and a proximal portion 212, separated by a shoulder 214. The metaphyseal element, the diaphysis, and the cement are not illustrated in FIG. 3. The shoulder 214 is adapted to come into contact against the exterior surface of the diaphysis. In this way, the shoulder 214 forms a blocking stop against the introduction of the rod 210 into the diaphyseal cavity, assuring that the rod 210 does not become embedded too deeply into the diaphyseal cavity, according to embodiments of the present invention.

The proximal portion 212 includes longitudinal grooves 213 adapted to cooperate with corresponding grooves (not shown) in the coupling cavity 26 or 126 in the metaphyseal element 20 or 120. In this way, the adjustment of the angular positioning and the relative locking in rotation are facilitated, according to embodiments of the present invention.

According to an alternative embodiment of the present invention, the proximal portion 212 includes other mechanisms for locking of the rotation of the metaphyseal element 20, 120 with respect to the diaphyseal element 200, for example cannulations, teeth and/or surface bumps, and the cavity of the metaphyseal element may include corresponding rotation locking mechanisms. According to another alternative embodiment of the present invention, the diaphyseal element 210 includes a different combination of mechanisms for rotation locking and translation locking.

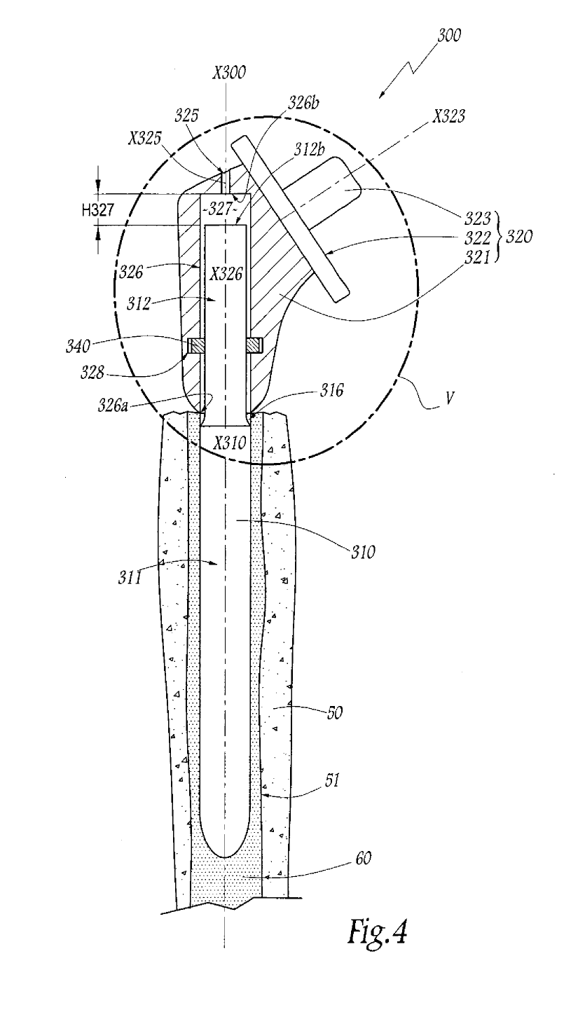

FIGS. 4 to 6 illustrate another modular prosthesis 300, according to embodiments of the present invention. Prosthesis 300 is adapted to be positioned in the same bone 50 as prostheses 1 and 100, according to embodiments of the present invention.

Certain elements forming the prosthesis 300 are similar to the elements forming the prosthesis 1, described above, and include the same reference numerals increased by 300. This includes the cylindrical rod 310 with axis X310 including a distal portion 311 and a proximal portion 312 with an end 312b, the metaphyseal element 320, the support surface 322, the mount 323 with axis X323, the injection canal 325 with axis X325, the coupling cavity 326 with axis X326, the mouth 326a and interior surface 326b, the reception chamber 327, the chamber 328, as well as the annular seal 340, according to embodiments of the present invention.

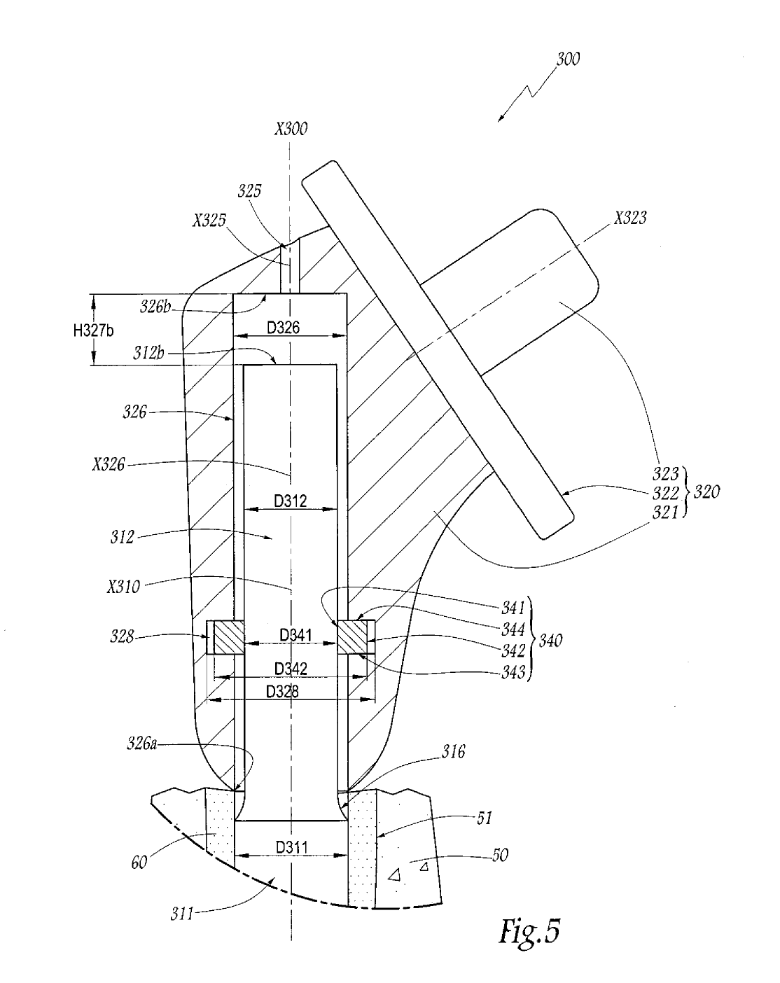

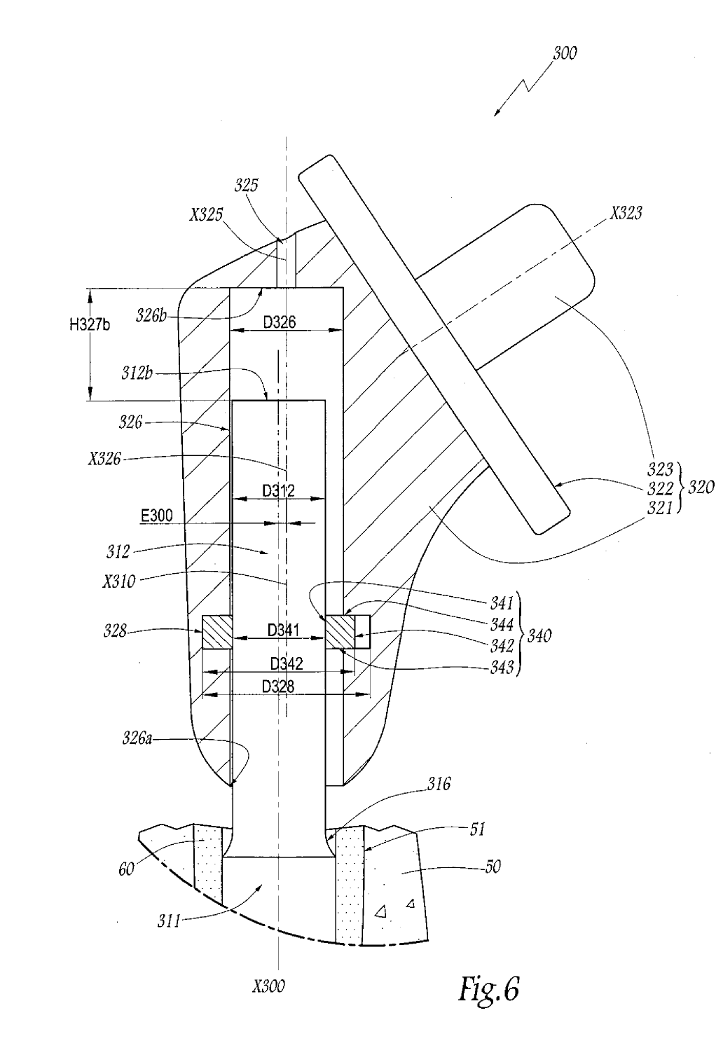

The differences may be found at the interface between the rod 310 and the metaphyseal element 320, particularly with respect to the proximal portion 312, the cavity 326, the chamber 327, the chamber 328, and the seal 340. A reduction of the diameter 316 is shown on the rod 310, between the distal portion 311 which has a diameter D311 and the proximal portion 312 which has a diameter D312 smaller than diameter D311. Contrary to the situation for prosthesis 1 in which the proximal portion 12 of the rod 10 penetrates into the coupling cavity 26, with sliding of the complementary cylindrical surfaces, the prosthesis 300 includes a transverse annular space between the proximal portion 312 and the cavity 326. In other words, there is some “play” between the proximal portion 312 and the cavity 326. As such, the axes X310 and X326 are aligned in FIGS. 4 and 5, and offset transversally in FIG. 6.

The annular seal 340 includes an interior diameter 341 which is initially smaller than diameter D312, a cylindrical exterior surface 342 of diameter D342, an annular surface 343 facing the distal portion 311, and an annular surface 344 facing the injection canal 325, according to embodiments of the present invention. The seal 340 deforms as the proximal portion 312 is introduced in sliding within the diameter 341, such that the diameter D341 is substantially equal to the diameter D312 once the seal 340 is positioned on the rod 310, according to embodiments of the present invention. The surfaces 343 and 344 are showed sliding perpendicularly to the axis X326 in the cavity 328, which has an interior diameter D328, which is larger than diameter D342. In this way, the seal 340 forms a watertightness for the chamber 327 and the cavity 326 for the side oriented towards the diaphysis 50, while being mobile, on one hand, perpendicularly to the axis X326 in the chamber 328, and on the other hand, axially following axis X310 in staying coupled with the metaphyseal element 320 and in sliding on the proximal portion 312 of the rod 310. According to an alternative embodiment of the present invention (not shown), the rod 310 does not have a transition to a smaller diameter at location 316, but instead the cavity 326 has a diameter D326 which is larger than the diameter D312 of the proximal portion 312, so as to make a space between them transversally to the axis X300, according to embodiments of the present invention. According to another alternative embodiment of the present invention, the rod 310 and/or the cavity 326 include other geometric configurations adapted to suit the desired interaction between the rod 310 and the cavity 326.

In practice, as with prostheses 1 and 100, the rod 310 is first inserted into the diaphyseal cavity 51, then locked in its final configuration by injecting cement or resin 60 in liquid state into the cavity 51. Then, after curing of the resin 60, the metaphyseal element 320 is positioned by the surgeon on the rod 310, which serves as intermediate support between the metaphyseal element 320 and the bone 50. Particularly, the proximal portion 312 of the rod 310 penetrates into the coupling cavity 326 and slides in the inner diameter 341 of the seal 340 which has been prepositioned in the cavity 328. In this way, the chamber 327, which is defined by the walls of the cavity 326, the surface 344 of the seal 340, and the proximal portion 312, has a variable volume.

At this stage, the height of the prosthesis 300 can be adjusted by progressively injecting a fluid (not shown), via the injection canal 325 which opens at the level of the surface 326b, into the reception chamber 327. The rod 310 is rigidly fixed in the diaphyseal cavity 51, while the metaphyseal element 320 can move relatively to the proximal portion 312. Under the effect of the internal pressure of the chamber 327, exerted by the fluid in the cavity 326 and the proximal portion 312, more particularly between the surfaces 312b and 326b, the volume of the chamber 327 increases.

In particular, the reception chamber 327 has a dimension H327b which is variable along the longitudinal direction of the prosthesis 300. The dimension H327b can be measured along the axis X300 between the surfaces 312b and 326b. In other words, the dimension H327b of the reception chamber 327 is variable along the axis X300, as a function of the quantity of fluid admitted into the chamber 327. Accordingly, while the fluid is injected, the height of the prosthesis 300 progressively increases until attaining the desired position.

Additionally, as illustrated in FIG. 6, the metaphyseal element 320 can be shifted laterally with respect to the rod 310 in order to better position the prosthesis 300. In this case, a transverse offset E300 is measured in a plane perpendicular to the axes X310 and X326. The seal 340 is then displaced within the cavity 328, such that the inner diameter 341 maintains the watertightness and/or seal on the proximal portion 312, while the surfaces 343 and 344 travel along a radius established by the diameter D342, at the end of the chamber 328, according to embodiments of the present invention. According to an alternative embodiment of the present invention (not shown), the chamber 328 has a diameter D328 more significant, in which case the surface 342 does not come into abutment with the end of the chamber 328, but the proximal portion 312 comes into contact with the cylindrical wall of the cavity 326.

In other words, the chamber 327 has a volume that is variable as a function of the length of the rod 310 inserted into the cavity 326 and of the quantity of the fluid introduced into the chamber 327. For a given internal volume, the chamber 327 has a variable configuration as a function of the transverse offset E300 between the axes X312 and X326.

According to some embodiments of the present invention, the fluid is an agglutinant fluid. In this way, the relative positioning between the elements 310 and 320 is variable and can be adjusted with precision, before, during, or after injection of the fluid. This positioning is axially variable along the longitudinal axis X300 of the prosthesis and/or transversally at the longitudinal axis X300 and/or angularly about the longitudinal axis X300, according to embodiments of the present invention.

According to an alternative embodiment of the present invention (not shown), the prosthesis includes several chambers for receiving agglutinant fluid. The chambers formed at the interior of the prosthesis may be isolated from each other, such that each chamber can be filled independently from the other chambers. For example, the chambers can be defined by separation walls which are radial or concentric with respect to the longitudinal axis of the prosthesis. The different chambers can have different configurations. According to one non-limiting example, a first chamber can be filled with fluid during the initial placement of the prosthesis, then a second chamber can be filled during resumption of the prosthesis in order to modify the relative positioning of the metaphyseal and diaphyseal elements.

In practice, the modular prosthesis permits the repair of a fracture, without requiring instrumentation that is cumbersome and difficult to manipulate to adjust the prosthesis, in particular for adjusting the relative positioning between the metaphyseal and diaphyseal elements. Accordingly, the prosthesis permits a freedom from ancillary and trial implants. The prosthesis can be easily adjusted in height and/or laterally and/or angularly, then can be rendered unitary and locked in place with the help of agglutinant fluid when the adjustment is satisfactory, without it being necessary to use data from multiple preliminary trials performed on the prosthesis, according to embodiments of the present invention.

According to embodiments of the present invention, the prosthesis can be implemented in the particular context of a surgical procedure and/or in the context of any post-operative application. In this case, the surgeon desolidifies the diaphyseal element and the metaphyseal element by introducing fluid under pressure into the reception chamber. For example, the prosthesis can be configured to receive a syringe for fluid injection. The fluid can be an agglutinant fluid, and/or a disagglutinant fluid, which permits the repositioning of the elements of the prosthesis before proceeding with a new injection of agglutinant fluid, according to embodiments of the present invention.

Throughout the present disclosure, the technical characteristics illustrated in various figures can be, in totality or for certain ones, combined between the various embodiments. Also, the prosthesis can be adapted to the particular needs of the surgeon. The prosthesis can be implemented with any type of metaphyseal element, anatomical or reversed.

In practice, the surgeon can make use of a surgical kit including different modular prosthetic elements. For example, one such modular prosthetic kit includes different metaphyseal elements corresponding to different head heights and/or different cavity configurations. Alternatively, the kit may include different diaphyseal rods, corresponding to different heights and/or different proximal rod diameters. Finally, the kit could include a container including a given quantity of agglutinant fluid, sufficient for implantation of at least one prosthesis. The kit is available to the surgeon in the form of a box, a set or packaging, known to the surgeon, of which the arrangement can be previewed to facilitate the location of the modular elements and to save the surgeon time. Different kits may be used, including varying numbers of modular elements, either reduced and thus less expensive, or important and thus adapted to a large number of cases, according to embodiments of the present invention. With such a modular prosthetic kit, the surgeon can be ready to rapidly face any situation which arises during the surgical operation.

Various modifications and additions can be made to the exemplary embodiments discussed without departing from the scope of the present invention. For example, while the embodiments described above refer to particular features, the scope of this invention also includes embodiments having different combinations of features and embodiments that do not include all of the described features. Accordingly, the scope of the present invention is intended to embrace all such alternatives, modifications, and variations as fall within the scope of the claims, together with all equivalents thereof.

What is claimed is:

1. A modular prosthesis comprising:
a diaphyseal element adapted for implantation into a bone;
a metaphyseal element;
a fluid reception chamber formed between the diaphyseal element and the metaphyseal element, the fluid reception chamber comprising a proximal end, a distal end, and a side wall; and
an injection canal in fluid communication with the fluid reception chamber;
wherein the fluid reception chamber is configured to receive fluid injected via the injection canal, such that a length of the modular prosthesis is adjustable in a longitudinal dimension based on a volume of fluid received by the fluid reception chamber.

2. The modular prosthesis of claim 1, wherein the metaphyseal element comprises a mount, the mount adapted to receive a prosthetic articulating surface.

3. The modular prosthesis of claim 1, wherein one of the proximal end and the distal end is formed by the diaphyseal element, and wherein the other of the proximal end and distal end is formed by the metaphyseal element.

4. The modular prosthesis of claim 1, wherein the side wall is formed by the metaphyseal element.

5. The modular prosthesis of claim 4, wherein the proximal end is formed by the metaphyseal element.

6. The modular prosthesis of claim 1, wherein the injection canal is substantially cylindrical and formed about a canal axis, wherein the fluid reception chamber is substantially cylindrical and formed about a chamber axis, and wherein the chamber axis and canal axis are substantially coaxial.

7. The modular prosthesis of claim 1, wherein the injection canal is formed in the metaphyseal element.

8. The modular prosthesis of claim 1, wherein the diaphyseal element comprises a proximal end and a distal end, wherein the proximal end of the diaphyseal element is substantially cylindrical and has an outer diameter, wherein the side wall is substantially cylindrical and has an inner diameter, wherein the outer diameter is larger than the inner diameter.

9. The modular prosthesis of claim 1, wherein the metaphyseal element is adjustable relative to the diaphyseal element along a transverse dimension perpendicular to the longitudinal dimension, prior to hardening of the fluid.

10. The modular prosthesis of claim 1, wherein the metaphyseal element is rotatably adjustable relative to the diaphyseal element about an axis substantially parallel to the longitudinal dimension, prior to hardening of the fluid.

11. The modular prosthesis of claim 1, wherein the diaphyseal element comprises a proximal portion and a distal portion, wherein the distal portion is implanted into the bone, wherein the metaphyseal element comprises the side wall, wherein the side wall comprises a seal cavity, the modular prosthesis further comprising:
a seal housed by the seal cavity, an inner surface of the seal conforming to an outer surface of the proximal portion of the diaphyseal element, wherein the proximal portion slides longitudinally with respect to the seal while the seal substantially prevents escape of the fluid from the fluid reception chamber beyond the seal.

12. The modular prosthesis of claim 11, wherein the seal cavity has a longitudinal height, wherein the seal substantially fills the longitudinal height, wherein the seal cavity has an inner transverse dimension larger than an outer transverse dimension of the seal, such that the seal maintains a fluid barrier when the metaphyseal element is adjusted relative to the diaphyseal element along a transverse dimension perpendicular to the longitudinal dimension, prior to hardening of the fluid.

13. The modular prosthesis of claim 1, wherein the diaphyseal element comprises a proximal portion and a distal portion, wherein the distal portion is implanted into the bone, and wherein the proximal portion interlocks with the metaphyseal element to substantially inhibit rotation of the metaphyseal element with respect to the diaphyseal element about the longitudinal dimension.

14. The modular prosthesis of claim 1, wherein the diaphyseal element comprises a shoulder located between its proximal and distal ends.

15. A method for implanting a modular prosthesis, comprising:
inserting an agglutinating fluid progressively into a fluid reception chamber formed between a first prosthesis element and a second prosthesis element in order to adjust a position of the first prosthesis element with respect to the second prosthesis element along a longitudinal dimension; and
permitting the agglutinating fluid to agglutinate to rigidly fix positioning of the first prosthesis element with respect to the second prosthesis element.

16. The method of claim 15, wherein a first volume of the fluid reception chamber is variable, and wherein inserting the agglutinating fluid progressively into the fluid reception chamber comprises increasing at least one dimension of the fluid reception chamber based on a second volume of agglutinating fluid inserted into the fluid reception chamber.

17. The method of claim 15, further comprising adjusting the position of the first prosthesis element with respect to the second prosthesis element along a transverse dimension substantially perpendicular to the longitudinal dimension prior to agglutination of the agglutinating fluid.

18. The method of claim 16, further comprising adjusting the position of the first prosthesis element with respect to the second prosthesis element rotationally about an axis corresponding to the longitudinal dimension prior to agglutination of the agglutinating fluid.

19. The method of claim 15, wherein inserting the agglutinating fluid comprises inserting the agglutinating fluid progressively into the fluid reception chamber via an injection channel formed in one or more of the first prosthesis element and the second prosthesis element.

20. The method of claim 19, wherein the injection channel is formed in the first prosthesis element.

21. The method of claim 15, further comprising:
forming a bone cavity in a bone;
placing the second prosthesis element into the bone cavity; and
rigidly fixing the second prosthesis element to the bone cavity with surgical cement.

22. The method of claim 21, wherein rigidly fixing the second prosthesis element to the bone cavity is done before adjusting the position of the first prosthesis element with respect to the second prosthesis element.
